(19) United States
(12) Patent Application Publication
(10) Pub. No.: US 2003/0059941 A1
(43) Pub. Date: Mar. 27, 2003

(54) Transduced marrow stromal cells

(76) Inventors: Darwin J. Prockop, New Orleans, LA (US); Emily Schwarz, River Ridge, LA (US)

Correspondence Address: MORGAN, LEWIS & BOCKIUS LLP, 1701 MARKET STREET, PHILADELPHIA, PA, 19103-2921, US

(21) Appl. No.: 10/155,298

(22) Filed: May 24, 2002

Related U.S. Application Data
(60) Provisional application No. 60/298,150, filed on Jun. 14, 2001.

(51) Int. Cl. A61K 48/00; C12N 5/06; C12N 5/08; C12N 15/867

(52) U.S. Cl. 435/455; 435/456; 424/93.21; 435/372; 435/353

(57) ABSTRACT

The present invention embodies a method of transducing marrow stromal cells with retroviral vectors comprising the TH and GC enzyme precursors of L-DOPA. The invention also describes a method of producing exogenous L-DOPA using this transduction method. Novel retroviral vectors comprising TH and GC, with an intervening IRES are also described.

### CROSS REFERENCE TO RELATED APPLICATIONS

This application claims priority under 35 U.S.C. § 119(e) to U.S. Provisional Application No. 60/298,150, filed Jun. 14, 2001.

### STATEMENT REGARDING FEDERALLY SUPPORTED RESEARCH AND DEVELOPMENT

The invention was made in part using funds obtained from the U.S. Government (National Institutes of Health Grant Nos. AR47161 and A42210) and the U.S. government may have certain rights in the invention.

### BACKGROUND

Bone marrow stromal cells (MSCs) are stem cells from adult bone marrow that can give rise to both mesenchymal and non-mesenchymal lineages. MSCs provide feeder layers for cultures of hematopoietic precursors and can differentiate into osteoblasts, adipocytes, and myoblasts (Owen, M E, et al., Cell and Molecular Biology of Vertebrate Hard Tissues, Ciba Foundation Symposium, Chicester, UK, pp. 42-60, 1988; Caplan, A I, et al., J. Orthoped. Res.; 9:641-650, 1991; Bruder, S P, et al., J. Cell Biochem., 64:278-294, 1997; Prockop, D J, Science, 276:71-74, 1997). To a limited degree, MSCs may also migrate through the blood brain barrier to contribute to lineages of the CNS when transplanted systemically (Eglitis M A et al., Proc. Natl. Acad. Sci. USA, 94(8):4080-4085, 1997; Pereira R F et al., Proc Natl Acad Sci USA, 95:1142-1147, 1998). When transplanted into the adult rat brain, human MSCs survive for long periods of time, migrate in a manner similar to rat astrocytes, and do not elicit host inflammatory or immune responses (Azizi S A et al., Proc. Natl. Acad. Sci. USA; 95:3908-3913, 1998). Recently, the versatility of MSCs was further revealed by the observation that MSCs can generate cell lineages of the central nervous system (CNS). For example, murine MSCs transplanted into the paraventricular zone of neonatal mice displayed neuronal markers glial fibrillary acidic protein (GFAP) and neurofilament-L, indicative of differentiation to both astrocytes and neurons, respectively (Kopen G C et al., Proc Natl Acad Sci USA; 96(19)10711-10716, 1999). In vitro, as many as 80% of MSCs exhibited characteristics of neurons when incubated with a cocktail of antioxidants in the absence of serum (Woodbury D. et al., J Neurosci Res, 61:364-370, 2000). In another study, adult human and murine MSCs incubated with retinoic acid and brain-derived neurotrophic factor (BDNF) or co-cultured with fetal mesencephalic cells expressed some markers specific for neural cells in vitro (Sanchez-Ramos, J et al., Exp. Neurol, 164:247-256, 2000). Therefore, it would appear that MSCs may be useful in treating CNS disorders or diseases by transplantation of MSCs into the CNS.

For example, presently the mainstay of therapy for treatment of Parkinson's disease involves oral administration of L-3,4,-dihydroxyphenyalanine (L-DOPA). However, the effectiveness is variable among patients and decreases with time (Obeso, J A, et al., Eur J Neurosci, 6(6):889-897, 1994). Many in vivo and ex vivo strategies have been pursued to replace L-DOPA/dopamine in the denervated striatum. One strategy is to use viral vectors. For example, adeno-associated viral (AAV) vectors were successful in producing expression of the protein precursors are for L-DOPA tyrosine hydroxylase-2 (TH) and GTP Cyclohydrolase I (GC) genes in neurons of the striatum for up to 1 year (Mandel, R J et al., Exp Neurol, 159:47-64, 1999). Controlled delivery of L-DOPA was achieved by infusion into the striatum of adenovirus containing the TH gene driven by a tetracycline inducible promoter (Corti, O et al., Proc Natl Acad Sci USA, 96:12120-12125, 1999).

Another strategy is to use cells as vectors. Cellular gene therapy in the rat model of Parkinson's Disease was accomplished by using primary astrocytes retrovirally transduced with the TH gene driven by the astrocyte specific promoter for glial fibrillary acid protein (GFAP; Cortez, N et al., J Neurosci Res, 59:39-46, 2000). Also, primary fibroblasts from rats were transduced with the genes for TH and GC and transplanted into the denervated rat striatum (Bencsics, et al., J Neurosci, 16 (14):4449-4456, 1996). The fibroblasts continued to synthesize L-DOPA in vivo, but the production was short-lived. Still another strategy was to use neural stem cells differentiated into dopaminergic neurons by overexpression of the nuclear hormone receptor, Nurrl (Wagner J et al., Nat Biotechnol, 17(7):653-659, 1999).

However, many of the current strategies for Parkinson's therapy require either direct administration of active viral vectors or the use of fetal tissue and/or cells that can only be obtained by invasive procedures. Therefore, there is a strong need for a therapeutic regimen having minimal side effects and which does not require invasive procedures to treat Parkinson's Disease. It is evident that this invention satisfies the need for a more amenable therapy for Parkinson's Disease, and many other CNS diseases and disorders.

### SUMMARY OF THE INVENTION

The invention includes a method for transducing marrow stromal cells. The method comprises infecting marrow stromal cells with a vector which comprises a bicistronic coding region comprising a nucleic acid encoding tyrosine hydroxylase type I (TH) and a sequence encoding GTP cyclohydrolase I (GC), operably linked to a promoter/regulatory region, thereby transducing the marrow stromal cell.

In a preferred embodiment of the invention, the vector is a virus or a plasmid. More preferably, the virus is a retrovirus. Even more preferably, the retrovirus is self-inactivating.

In another preferred embodiment, the nucleic acid encoding TH and the nucleic acid encoding GC are separated by an internal ribosomal entry site (IRES).

In yet another preferred embodiment, the marrow stromal cell is a human marrow stromal cell or a rat marrow stromal cell.

The invention also includes a method of treating Parkinson's disease. The method comprises administering to a patient marrow stromal cells transduced by the method described above, wherein the administration of the marrow stromal cells alleviates symptoms of Parkinson's disease.

The invention further includes a method of treating a disease characterized by a deficiency in 3,4-dihydroxyphenylalanine (L-DOPA). The method comprises administering to a patient a marrow stromal cell transduced by the method described above, wherein the administration of the marrow stromal cells regulates the production of L-DOPA causing alleviation of symptoms of said disease.

The invention further includes a method for producing exogenous L-DOPA. The method comprises transducing a marrow stromal cell by the described above and expressing tyrosine hydroxylase type I (TH) and GTP cyclohydrolase I (GC) in the marrow stromal cell thereby producing exogenous L-DOPA.

The invention also includes a vector comprising a nucleic acid encoding TH and GC separated by an internal ribosomal entry site (IRES).

In a preferred embodiment, the vector comprises a promoter sequence operably linked to the nucleic acid encoding TH and GC. More preferably, the promoter sequence is a cytomegalovirus promoter, phosphoglycerate kinase-1 promoter, or human histone H4.

In another preferred embodiment, the vector is retroviral. Even more preferably, the vector is a self-inactivating retrovirus.

In yet another preferred embodiment, the vector can be murine leukemia viral vector (LXSN), murine stem cell viral vector (MSCV) or a self-inactivating retroviral vector, wherein the self-inactivating retroviral vector further comprises a cytomegalovirus or phosphoglycerate kinase promoter.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiment(s) which are presently preferred. However, it should be understood that the invention is not limited to the precise arrangements and instrumentalities shown. In the drawings:

FIG. 1, comprising FIGS. 1A-1D, is a set of graphs depicting relative promoter strength in rMSCs. FIGS. 1A, 1B, and 1C represent the percentage of rMSCs expressing GFP relative to the control sample. FIG. 1A represents cells transfected with CMV-GFP plasmid, FIG. 1B represents cells transfected with PGKGFP plasmid, and FIG. 1C represents cells transfected with H4-GFP plasmid. FIG. 1D is a graph depicting GFP expression 48 hours post-transfection. Data represent mean±standard deviation.

FIG. 2, comprising FIGS. 2A-2D, is a set of schematic representations of retroviral diagrams. FIG. 2A is a diagram of the standard Maloney murine leukemia viral vector (LXSN), comprising the TH and GC genes, separated by an internal ribosome entry site (IRES), the whole TH-IRES-GC being labeled TIG. FIG. 2B is a diagram of murine stem cell vector (MSCV) comprising TIG. FIG. 2C is a self-inactivating retroviral vector with the mouse phosphoglycerate kinase-1 (PGK) promoter driving expression of TIG. FIG. 2D is a diagram of a self-inactivating retroviral vector with the CMV promoter driving expression of TIG. “Neo” represents neomycin resistance gene, 3′delLTR represents the 3′ long terminal repeat with deleted enhancer sequences, 5′LTR represents the 5′ long terminal repeat, and “psi” represents the packaging signal.

FIG. 3, comprising FIGS. 3A-3I, is a set of images (FIGS. 3A-3H) and a graph (FIG. 3I) depicting expression of GFP and production of L-DOPA in transfected Phoenix-amphotropic packaging cells 48 hours post-transfection. FIGS. 3A, 3C, 3E, and 3G represent phase-contrast microscopy images of the packaging cells transfected with LXSN-GFP, MSCV-GFP, pSIR-CMV-GFP, and pSIR-PGK-GFP, respectively. FIGS. 3B, 3D, 3F, and 3H represent fluorescence microscopy of expression of GFP in the same field of cells corresponding to FIGS. 3A, 3C, 3E, and 3G, respectively. FIG. 3I is a bar graph depicting L-DOPA production in packaging cells transfected with retroviral vectors comprising either TH or the bicistronic TH-IRES-GC sequence, 48 hours post transfection. Data is represented as mean and standard deviation.

FIG. 4, comprising FIGS. 4A-4H, is a set of images representing GFP expression in rMSCs infected with either LXSN-GFP (FIGS. 4A and 4B), MSCV-GFP (FIGS. 4C and 4D), pSIR-PGK-GFP (FIGS. 4E and 4F), or pSIR-CMV-GFP (FIGS. 4G and 4H). Expression of the same field was analyzed 4 days post-infection using phase-contrast microscopy (FIGS. 4A, 4C, 4E, and 4G) and fluorescence microscopy (FIGS. 4B, 4D, 4F, and 4H). Magnification is 200×.

FIG. 5, comprising FIGS. 5A, 5B, and 5C, is a trio of graphs demonstrating GFP expression flow cytometry in stably transduced rMSCs. FIG. 5A represents cells transduced with LXSN-GFP, FIG. 5B represents cells transduced with MSCV-GFP, and FIG. 5C represents cells transduced with pSIR-PGK-GFP.

FIG. 6, comprising FIGS. 6A-6G, is a set of images depicting TH immunostaining and western blot analysis of stably transduced rMSCs. FIGS. 6A and 6B depict rMSCs transduced with LXSN-TIG and stained with DAPI (FIG. 6A) to detect viable cells or immunostained to detect TH (FIG. 6B). FIGS. 6C and 6D depict rMSCs transduced with MSCV-TIG and stained with DAPI (FIG. 6C) or immunostained to detect TH (FIG. 6D). FIGS. 6E and 6F depict rMSCs transduced with pSIR-PGK-TIG and stained with DAPI (FIG. 6E) or immunostained to detect TH (FIG. 6F). Magnification is 200×. FIG. 6G is an image representing expression of TH-trans-protein in whole cell lysates from transduced rMSCs. Lane 1 is a positive control of rMSCs modified with a retrovirus having TH driven by the CMV promoter; Lane 2 is untransduced rMSCs; Lane 3 is rMSCs MSCV-TH only; Lane 4 is rMSCs MSCV-TIG; Lane 5 is rMSCs LXSN-TIG, and Lane 6 is rMSCs pSIR-PGK-TIG.

FIG. 7 is a graph depicting L-DOPA production in high-density cultures of stably transduced rMSCs. Data represent mean±standard error of the mean, n=3.

FIG. 8 is a graph depicting L-DOPA production in low-density cultures of stably transduced rMSCs. Data represent mean±standard error of the mean, n=3.

### DETAILED DESCRIPTION

The invention herein described relates to methods of transducing marrow stromal cells (MSCs) and uses for marrow stromal cells so transduced. Marrow stromal cells may be transduced for production of neuronal proteins or protein precursors, and later transplanted into central nervous system tissue to alleviate and/or treat symptoms of neuronal diseases.

MSCs are isolated from a patient's bone marrow, transduced with a recombinant vector that expresses a desired protein or protein precursor, and transplanted back into the CNS of the same patient. MSCs from a single aspirate can produce up to 10¹³ MSCs in about 6 weeks (Colter, et al., Proc Natl Acad Sci USA, 97(7):3213-3218, 2000). Thus, adequate numbers of MSCs are readily obtained for most therapeutic purposes, making MSC therapy a vast improvement over the current therapies.

Autologous bone marrow stromal cells can be engineered to produce a variety of neuronal proteins associated with many neuronal diseases. Briefly, in an embodiment of the present invention, marrow stromal cells are transduced with viral vectors comprising a promoter element and a bicistronic sequence comprising the nucleic acid corresponding to the protein precursors of L-DOPA, the tyrosine hydroxylase-2 (TH) enzyme and the GTP cyclohydrolase I (GC) enzyme, and an internal ribosomal entry site (IRES) intervening between each of the TH and GC cistrons. The MSCs so transduced are transplanted into the central nervous system and begin producing the TH and GC proteins, and ultimately, L-DOPA, in vivo, thus aiding the patient in producing the necessary L-DOPA to effect prevention, arrest of disease progression, or therapeutic relief. In a particularly preferred embodiment, the MSCs are human MSCs. While these are the preferred embodiments, this method of the invention is intended to be used with other mammals having an L-DOPA deficient condition.

In one embodiment, the viral vector used to transduce the marrow stromal cells is a retroviral vector, and preferably, a self-inactivating retroviral vector. The use of self-inactivating retroviruses in gene therapy is a relatively simple and effective way to integrate therapeutic proteins into marrow stromal cells. Control of the expression of such therapeutic proteins occurs via an internal promoter. Viral transcripts are not detected in target cells and thus preparing self-inactivating retroviruses is safe (Nakajima, K et al., FEBS, 315(2):129-133, 1993). Recently, a self-inactivating lentiviral vector encoding glial-derived neurotrophic factor (GDNF) driven by the PGK promoter was used in a rat model of Parkinson's Disease (Deglon, N et al., Hum Gene Ther, 11:179-190, 2000). In that study, in vivo expression of GDNF was exhibited for up to 14 weeks and no adverse effects of viral transduction on the host brain were observed. Therefore, self-inactivating retroviral vectors are preferred when practicing the method of the invention.

The promoter of the viral vector can be any promoter useful in effecting expression in the selected virus, including, but not limited to, phosphoglycerate kinase-3 (PGK), cytomegalovirus (CMV), or Histone4 (H4). A preferred vector for the insertion of the TH-IRES-GC bicistronic sequence is the combination of a self-inactivating retroviral vector and either the PGK or the CMV promoter. Other preferred vectors for inserting the TH-IRES-GC sequence include the standard Maloney murine leukemia viral vector (LXSN), which comprises the SV40 promoter, and the murine stem cell viral vector (MSCV), which comprises PGK as its internal promoter.

The invention also encompasses a method of treating diseases characterized by deficiency in dopamine including, but not limited to, Parkinson's disease, as well as 6-pyruval-tetrahydropterin synthase deficiency (Dudesed, A. et al., Eur. J. Pediatrics, 160(5):267-276, 2001).

It has been suggested that L-DOPA-producing MSCs that are plated at low-density are a superior option to cells plated densely to engraft into the brain as they might resemble a more primitive state of the MSCs (Colter, et al., Proc Natl Acad Sci USA, 97(7):3213-3218, 2000.). MSCs may be easily isolated from a small sample of bone marrow, engineered in culture to produce L-DOPA, expanded to generate L-DOPA-producing cells reaching a trillion in number, and the appropriate number of therapeutic MSCs could be transplanted back into the striatum of the same patient. Therefore, it is further preferred that the transduced MSCs be plated at a low density for optimal proliferation in vitro before transplantation.

Methods of treating an animal by transplanting MSCs into the central nervous system are described in WO 96/30031 and WO 99/43286, which are incorporated by reference as if set forth in their entirety herein. Methods of administering MSCs to a patient are identical to those used for MSCs as described in WO 96/30031 and WO 99/43286. The methods encompass introduction of an isolated nucleic acid encoding a therapeutic protein into MSCs and also encompass using MSCs themselves in cell-based therapeutics where a patient is in need of the administration of such cells. The differentiated neural cells are preferably administered to a human, and further, the MSCs are preferably administered to the central nervous system of the human. In some instances, the MSCs are administered to the corpus striatum portion of the human brain. The precise site of administration of MSCs will depend on any number of factors, including but not limited to, the site of the lesion to be treated, the type of disease being treated, the age of the human and the severity of the disease, and the like. Determination of the site of administration is well within the skill of the artisan versed in the administration of cells to mammals.

The mode of administration of the MSCs to the central nervous system of the human may vary depending on several factors including but not limited to, the type of disease being treated, the age of the human, whether the MSCs have isolated DNA introduced therein, and the like. Generally, cells are introduced into the brain of a mammal by first creating a hole in the cranium through which the cells are passed into the brain tissue. Cells may be introduced by direct injection, by using a shunt, or by any other means used in the art for the introduction of compounds into the central nervous system.

One skilled in the art would appreciate based on the disclosure presented herein that the number of MSCs administered to the patient may also vary depending on the patient and mode of delivery. Preferably, approximately fourteen million MSCs are administered per 70 kg patient. The skilled artisan would further appreciate that effective treatment may require repeat administration of MSCs to the patient approximately. Preferably, administration is repeated approximately once every six months.

As is well known in the art, MSCs can be obtained from a wide range of donor types. Preferably, MSCs are derived from the same patient to whom they will be administered. However, the invention should not be construed to be limited to administration of MSCs obtained from the patient, as MSCs from unmatched donors as well as other mammals is foreseeable. In cases in which patients receive MSCs other than their own, immunosuppression of the patient using standard procedures well known in the art is required.

One skilled in the art would appreciate that the levels of L-DOPA expression can be titrated using a regulatable expression system. Regulatable expression systems are characterized by the presence of a promoter whose ability to activate gene expression is inducible. In a preferred example, the tetracycline-inducible system which uses a tetracycline-inducible promoter to activate expression of L-DOPA can be used together with administration of tetracycline to the patient to regulate L-DOPA expression levels.

The levels of L-DOPA in a patient can be measured using standard techniques known in the art, including but not limited to Positron Emission Tomography (PET) in combination with isotopically-labeled analogs of L-DOPA.

Definitions

The articles “a” and “an” are used herein to refer to one or to more than one (i.e. to at least one) of the grammatical object of the article. By way of example, “an element” means one element or more than one element.

The term “alleviate” is used interchangeably with the term “treat”. As used herein, a symptom of an L-DOPA deficiency disorder is “alleviated” or “treated” if the severity and/or frequency of the symptom is reduced. A “therapeutic” treatment is a treatment administered to a subject who exhibits signs of pathology for the purpose of diminishing or eliminating those signs.

As used herein, “central nervous system” should be construed to include brain and/or the spinal cord of a mammal. The term may also include the eye and optic nerve in some instances.

As used herein, “bone marrow stromal cells”, “marrow stromal cells”, “isolated marrow stromal cells”, and “MSCs” are used interchangeably and are meant to refer to the small fraction of cells in bone marrow which can serve as stem cell-like precursors of osteocytes, chondrocytes, and adipocytes and which are isolated from bone marrow by their ability adhere to plastic dishes. Marrow stromal cells may be derived from any animal. In some embodiments, stromal cells are derived from rodents, and in others, primates, preferably humans.

The term “L-DOPA deficiency” should be construed to mean expression of less than normal levels of L-DOPA. A disease characterized by L-DOPA deficiency results from a decrease or a complete lack of L-DOPA expression.

As used herein, the term “disease” is construed to mean disease, disorder, or condition. A “disease” can be treated or prevented by the presence of a protein or proteins which alleviate, reduce, prevent or cause to be alleviated, reduced or prevented, the causes and/or symptoms that characterize the disease. Diseases which can be treated with a beneficial protein include diseases characterized by a gene defect as well as those which are not characterized by a gene defect but which nonetheless can be treated or prevented by the presence of a protein which alleviates, reduces, prevents or causes to be alleviated, reduced or prevented, the causes and/or symptoms that characterize the disease.

The term “cistron” should be construed to refer to a nucleic acid sequence, or segment, or fragment which has been purified from the sequences which flank it in a naturally occurring state, e.g., a DNA fragment which has been removed from the sequences which are normally adjacent to the fragment e.g., the sequences adjacent to the fragment in a genome in which it naturally occurs. The term also applies to nucleic acids which have been substantially purified from other components which naturally accompany the nucleic acid, e.g., RNA or DNA or proteins which naturally accompany it in the cell. Similarly, a “multicistronic” vector is one which comprises more than one cistron.

As used herein, “transfected cells” is meant to refer to cells to which a gene construct has been provided using any technology used to introduce nucleic acid molecules into cells such as, but not limited to, classical transfection (calcium phosphate or DEAE dextran mediated transfection), electroporation, microinjection, liposome-mediated transfer, chemical-mediated transfer, ligand mediated transfer or recombinant viral vector transfer.

The term “transduction” or “transduced cells” are used interchangeably herein and is used to define cells that have been infected with a virus. A method of transduction comprises infecting a cell with a virus comprising an isolated nucleic acid of interest, collected from the supernatant of different cells previously transfected with the same virus. The virus multiplies and is secreted into the medium in which the transfected cell is grown. The virus is collected and is introduced into a different cell population, whereby the viral nucleic acid is expressed.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### EXAMPLES

The invention is now described with reference to the following Example. This Example is provided for the purpose of illustration only, and the invention is not limited to this Example, but rather encompasses all variations which are evident as a result of the teaching provided herein.

In order to transplant MSCs that produce exogenous L-DOPA for long periods of time in vivo, the effects of various promoter elements on expression of TH and GC in the MSCs in vitro were studied. Previously, MSCs were engineered to express TH, the rate-limiting enzyme in dopamine biosynthesis, and GC, the enzyme providing the tetrahydropterin cofactor for TH (Schwarz E J et al., Hum Gene Ther, 10:2539-2549, 1999) by transfection with 2 vectors, one comprising the TH enzyme and one comprising the GC enzyme. The genetically engineered MSCs proved to synthesize and release L-DOPA. When the MSCs that synthesized L-DOPA were transplanted into the rat model of Parkinson's Disease, the L-DOPA was converted to dopamine metabolites, and behavioral recovery was observed; however, the ameliorative effect of transplanted MSCs was short-lived, presumably due to inactivation of transgenes introduced into the brain with retroviruses.

In a previous study, human MSCs transduced with a bicistronic vector encoding GFP and a selectable marker exhibited long-term expression of both genes in vitro for 6 months without any observed toxic effects to the MSCs (Marx J C, et al., Hum Gene Ther, 10:1163-1173, 1999). In the present invention, rMSCs transiently transfected with GFP downstream of the CMV and PGK promoters were able to express GFP (9 percent and 4 percent of cells, respectively). Similar relative patterns of reporter gene expression were seen when human MSCs were electroporated with constructs containing the chloroamphenicol acetyltransferase reporter gene controlled by either the CMV or PGK promoters (Keating A et al., Exp Hematol, 18:99-102, 1990).

MSCs were transduced sequentially with two separate retroviruses, each containing TH or GC driven by the CMV promoter. A 3.4 kb bicistronic construct comprising the TH gene and GC gene separated by an internal ribosome entry site (TH-IRES-GC) was created to avoid use of two separate retroviruses and minimize the need for expansion of engineered MSCs. A self-inactivating retroviral vector (pSIR) was also employed, in which a 3′ enhancer sequence in the LTR has been deleted, the 5′-LTR is inactivated upon integration into the target cell genome (Nakajima K et al., FEBS, 315(2):129-133, 1993), and the TH-IRES-GC central construct was driven by a promoter of choice. In addition, experiments to determine whether a small number of rMSCs producing L-DOPA could undergo a massive expansion in culture by a simple low-density plating were also completed. The materials and methods employed in these experiments are now described.

Construction of the Plasmids

The following represents a detailed description on production of each of the vectors and constructs used in the experiments described in this application. The generation of each vector and of each construct is described.

To obtain an ideal vector for gene transfer into MSCs in vitro, expression constructs consisting of either a cellular or viral promoter upstream of the reporter gene, enhanced green fluorescent protein (GFP), were prepared and the relative effectiveness of those promoters was tested in rMSCs transiently transfected with these vectors via calcium phosphate precipitation. The cytomegalovirus (CMV) promoter was used as a strong viral promoter, and the mouse phosphoglycerate kinase-1 (PGK) promoter and human histone 4 promoter (H4)²⁰ were used as promoters for housekeeping genes.

The plasmid EGFP-1 (Clontech, Palo Alto, Calif.) was digested with EcoRI and NotI to produce a 769 base pair fragment comprising enhanced green fluorescent protein (eGFP). This eGFP fragment was ligated into PCIneo (Promega, Madison, Wis.) digested with EcoRI and NotI, creating the P-CMV-GFP construct.

Similarly, the P-PGK-GFP was prepared by digesting the MSCVneo (Clontech, Palo Alto, Calif.) vector with BglII and PstI, filling in the PstI site to create a blunt end, and ligating the resulting 510 base pair fragment comprising the PGK promoter to an 851 base pair BglII to IpoI fragment of P-CMV-GFP, from which the CMV promoter was removed.

P-H4-GFP was generated by ligating an 800 base pair BamHI to EcoRV fragment comprising the Histone 4 promoter to the 851 base pair BglII to IpoI fragment of P-CMV-GFP lacking the CMV promoter (Hanly S M et al., Mol Cell Biol., 5(2):380-9, 1985).

Generation of Bicistronic Retroviral Vectors Expressing Both the TH and GC Genes

The human tyrosine hydroxylase type 2 (TH) gene was prepared by PCR amplification of a 1.5 kb sequence comprising TH cDNA from LNCX-TH as described in Bencsics C et al., J Neurosci, 16 (14):4449-4456, 1996, (kindly provided by Dr. U. J. Kang) using the following primers: Forward: 5′-CGCGATCGATTCCACACTGAGCCATGCC-3′ (SEQ ID NO: 1); Reverse: 5′-GCGCGATATCCTCCGGGACAGTGCAGAC-3′ (SEQ ID NO: 2). For all PCR, the reactions were initially denatured at 95 degrees Celsius for 2 minutes, subjected to 30 cycles of 95 degrees Celsius for 1 minute, 68 degrees Celsius for 30 seconds, and 73 degrees Celsius for 4 minutes, followed by a final extension at 73 degrees Celsius for 5 minutes. PFU polymerase (Stratagene, La Jolla, Calif.) was used in all PCR reactions.

The TH PCR product and the pBluescript KS(pBS) vector (Stratagene) were both digested with Clal and EcoRV. The TH PCR product was then cloned into the digested pBS vector to obtain the resultant pBS-TH construct.

The GTP cyclohydrolase I (GC) gene was amplified in a similar manner as that for TH. A 900 base pair sequence was amplified from the vector p-delta-gHCGC as described in Bencsics C et al., (provided by Dr. U. J. Kang) comprising rat GC cDNA using the following primers: Forward: 5′-CGCGGAATTCCCACAGGTCACGGCCGCC-3′ (SEQ ID NO: 3); Reverse: 5′-GCGCGGATCCGACAAGTATACCAACTGG-3′ (SEQ ID NO: 4). The PCR product and pBS were both digested with EcoRI-BamHI and the PCR product was cloned into the EcoRI to BamHI site of pBS to yield pBS-GC.

The mouse phosphoglycerate kinase-1 (PGK) promoter was obtained from the vector pPNT by amplifying a 511 base pair sequence with the following primers: Forward: 5′-CGCGCTCGAGAATTCTACCGGGTAG-3′ (SEQ ID NO: 5); Reverse: 5′-GCGCATCGATAGGTCGAAAGGCCCGGAG-3′ (SEQ ID NO: 6). The resultant PCR product and pBS were both digested with XhoI and ClaI and the PCR product was cloned into pBS to obtain pBS-PGK. Recombinant DNA in all instances was screened by restriction digest analysis and all positive clones were sequenced completely on an automated sequencer (Model 7700, ABI, Warrington, UK).

Since, as will be described later, both the CMV and PGK promoters were able to activate GFP expression in the rMSCs, the CMV and PGK promoters were used as internal promoters in the self-inactivating retroviral constructs. To avoid the use of two separate retroviruses, a bicistronic sequence was prepared comprising TH cDNA as the first cistron, an internal ribosome entry site (IRES), and GC cDNA as the second cistron (referred to as TH-IRES-GC, or TIG).

The bicistronic construct was made by digesting the pBS-GC vector with EcoRI and XbaI to create a 900 base pair fragment comprising GC. A separate digestion of the pBS-TH vector with NheI and EcoRI yielded a 1.5 kilobase pair fragment comprising TH. These fragments were ligated and then introduced into the NheI to XbaI site of PCIneo yielding PCIneo-THGC.

Next, the pIRESneo vector (Clontech Inc., Palo Alto, Calif. ) was digested with EcoRV and SmaI to yield a 900 base pair fragment comprising the encephalomyocarditis virus internal ribosomal entry site (IRES). The PCIneo-THGC was digested with EcoRV, and the IRES fragment was ligated into the PCIneo plasmid to arrive at the resultant vector, PCIneo-TH-IRES-GC. The central bicistronic construct of TH-IRES-GC is 3.4 kilobase pairs.

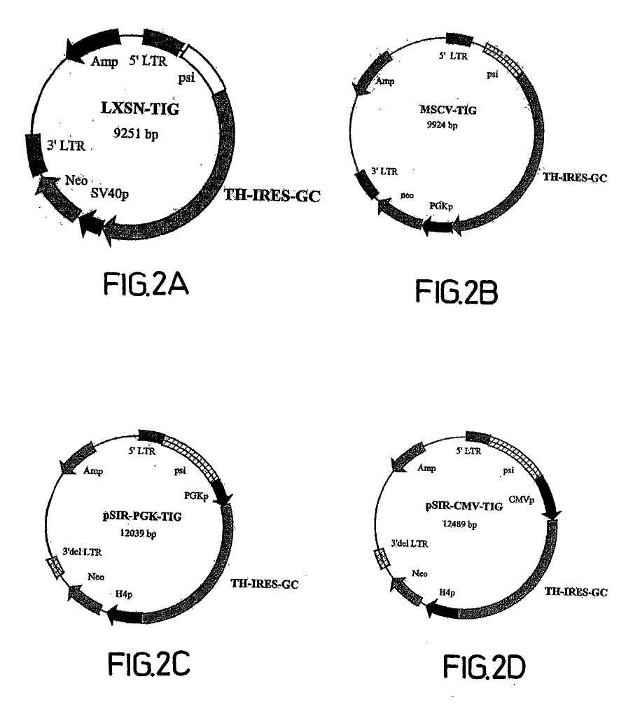

The 3.4 kilobase TH-IRES-GC (TIG) construct was then subcloned into four retroviral vectors: LXSN-TIG, MSCV-TIG, pSIR-PGK-TIG, and pSIR-CMV-TIG (FIG. 2). As a control for vector backbone and transduction efficiency, GFP was cloned into the four vectors in parallel, yielding the following vectors: LXSN-GFP, MSCV-GFP, pSIR-CMV-GFP, and pSIR-PGK-GFP.

The MSCV-GFP plasmid was prepared following the method described in 31. The MSCV-TH plasmid was prepared by digesting PCIneo-TH-IRES-GC with Clal (filled in to create a blunt end) and BamHI and ligating the resultant 1.5 kilobase pair fragment comprising TH cDNA into the MSCVneo digested with BglII and HpaI.

LXSN-GFP was prepared by the method disclosed in 32. MSCV-TH-IRES-GC and LXSN-TH-IRES-GC were constructed similarly using standard techniques (Ausubel, F. M., et al. 1987. In: Current Protocols in Molecular Biology, Greene Publisher Associates & Wiley Interscience, New York).

The self-inactivating retroviral vectors were prepared using a commercially available vector, pSIR (Clontech Inc., Palo Alto, Calif.). Plasmids comprising either PGK-TH-IRES-GC, PGK-GFP, CMV-TH-IRES-GC, or CMV-GFP cassettes were digested with BamHI and the resulting fragments were subcloned into the BamHI site of pSIR and transformed into XL-10 Gold cells (Stratagene; La Jolla, Calif.). Further details of plasmid construction are available upon request. All plasmids used for transfection were prepared with a plasmid kit (Maxiprep Kit; Qiagen, Valencia, Calif.).

Isolation and Culture of Primary Rat MSCs

Primary cultures of rMSCs were obtained from the femurs and tibias of adult male Lewis rats (Harlan, Indianapolis, Ind.) as described previously in Schwarz E J, et al., Hum. Gene Ther. 10:2539-2549, 1999. Briefly, rats were euthanized with a mixture of 70 percent CO₂ and 30 percent O₂. Tibias and femurs were removed and placed on ice in complete medium containing minimal essential medium with alpha modification (alpha-MEM; Gibco-BRL, Gaithersburg, Md.) with 20 percent fetal calf serum (Atlanta Biologicals, Norcross, Ga.), 2 millimolar L-glutamine, penicillin (100 units per milliliter), streptomycin (100 micrograms per milliliter), and amphotericin B (25 nanograms per milliliter; Mediatech, Herndon, Va.). Under sterile conditions, a 21-gauge needle attached to a 10 -milliliter syringe filled with medium was used to flush out the marrow. Bone marrow was filtered through a 70 micrometer nylon mesh and plated in a 75-square centimeter flask (Becton Dickinson, Franklin Lakes, N.J.). MSCs were isolated by their adherence to plastic.

About 24 hours after plating, non-adherent cells were removed and fresh medium was added. After the cells had grown to near confluency, they were passaged two to five times by being detached by trituration with 0.25 percent trypsin/1 millimolar EDTA for 5 minutes and replated at a density of around 5,000 cells per square centimeter.

Transfection of Phoenix Packaging Cells

Phoenix amphotropic packaging cells (derived from 293 cells) were obtained from the ATCC (Rockville, Md.) with permission of Dr. G. Nolan (Stanford University). The Stanford Registry Nos. for Phoenix Eco cells and Phoenix Ampho cells are SBR-422 and SBR-423, respectively. Phoenix cells were transfected with retroviral vectors by calcium phosphate precipitation as described in Pear W, et al., Proc Natl Acad Sci USA 90:8392-8396, 1993. PT67 cells (Clontech Inc., Palo Alto, Calif.) are also suitable for this purpose. Briefly, 24 hours prior to transfection, 2.5×10⁶ Phoenix cells were plated in 21.0 square centimeter plates in 5 milliliters of GM (10 percent heat-inactivated fetal bovine serum, 100 units per milliliter of penicillin, 100 units per milliliter of streptomycin, and 2 millimolar L-glutamine in Dulbecco's modified eagle's medium (DMEM)) and incubated at 37 degrees in 5% CO₂. Just prior to transfection, the medium was changed to GM containing 25 micromolar chloroquine.

The transfection cocktail was prepared by adding 500 microliters of 2× HEPES buffered saline solution (50 millimolar HEPES, pH 7.05; 10 millimolar KCl; 12 millimolar Dextrose; 280 millimolar NaCl; 1.5 millimolar Na₂HPO4) to 500 microliters of transfection mixture containing 10 micrograms of plasmid DNA in 240 millimolar CaCl₂. The transfection cocktail was added to the Phoenix cells, the cells were incubated at 37 degrees Celsius for 10 hours, and the medium was changed to fresh GM without chloroquine. GM was replaced 24 hours prior to viral harvest. Viral supernatants were collected 48 hours after the start of the transfection, filtered through a 0.45 micrometer filter, and stored at −80 degrees Celsius until further use. Phoenix cells were analyzed at the time of viral harvest for GFP expression and L-DOPA production as described.

Transient Transfection of rMSCs

rMSCs were plated at passage 3 at a density of 5,000 cells per square centimeter 24 hours prior to transfection in either 6 or 12 well plates. For 12 well plates (3.8 square centimeter wells), 1 microgram of plasmid DNA was incubated with 6 microliters of a cationic lipid reagent (GENEPORTER™; Gene Therapy Systems, San Diego, Calif.) in 0.5 milliliters of reduced-serum medium (OPTI-MEM™; Gibco-BRL). For 6 well plates (9.6 square centimeter wells), 2 micrograms of plasmid DNA and 12 microliters of GENEPORTER™ in 1 milliliter of OPTI-MEM™ were used. DNA and lipids were incubated in OPTI-MEM™ at room temperature for 30 minutes and the transfection mixture was added to the rMSCs, 500 microliters per well for 12 well dishes; 1 milliliter per well for 6 well dishes.

For control experiments, the PCIneo vector (Promega Corp., Madison, Wis.) alone was used in transfections. Cells were incubated with the transfection mixture for 5 hours at 37 degrees Celsius, after which an equal volume of complete medium containing 40 percent FCS was added to cells, yielding a final concentration of 20 percent FCS. The following day, fresh complete medium containing 20 percent FCS was added to the cells and cells were analyzed for GFP expression 72 hours after the start of the transfection.

Transduction of rMSCs

About 100,000 rMSCs were plated the day before infection in 21.0 square centimeter plates. At the time of infection, Day 1, 2.5 milliliters of complete medium containing 20 percent heat-inactivated FCS was added to the cells in the presence of 500 microliters of viral supernatant and 8 micrograms per milliliter of polybrene (Sigma, St. Louis, Mo.), and cells are incubated at 37 degrees in 5% CO₂. The infection procedure was repeated 24 hours later on Day 2. On Day 3, fresh complete medium was added with 20 percent FCS (not heat-inactivated). On Day 4, cells were split 1:2 in 55.0 square centimeter plates in complete medium containing 200 micrograms per milliliter of G418 for a period of 14 days. The surviving cells were pooled.

Analysis of GFP Expression

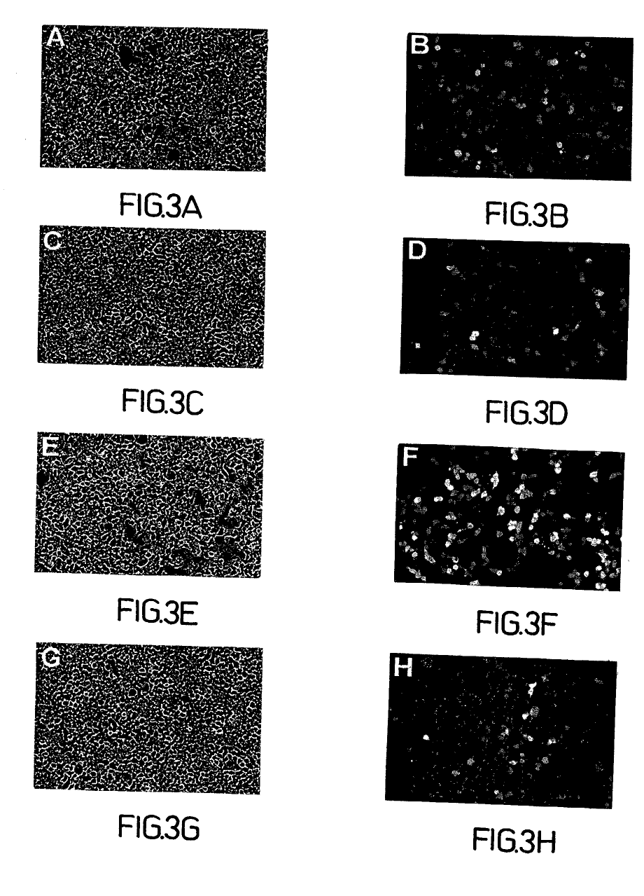

The retroviral constructs comprising GFP were tested for GFP expression and L-DOPA production, as well as the capacity to generate recombinant retrovirus. Different groups of Phoenix amphotropic packaging cells were transiently transfected with one of the vectors by calcium phosphate precipitation and analyzed for GFP expression by fluorescence microscopy (see FIG. 3) and flow cytometry. L-DOPA production was evaluated by transiently transfecting groups of Phoenix cells with one of the vectors. L-DOPA production in the media was analyzed by electrochemical detection and HPLC analysis. As a control, the MSCV vector containing the TH gene alone was used in the transfection.

Following transfection, rMSCs were washed three times with phosphate-buffered saline (PBS, pH 7.4) and the GFP signal was analyzed with a fluorescent plate reader (Cytofluor II, PerSeptive Biosystems, Framingham, Mass.) using a 485/20-nanometer excitation filter and a 530/30-nanometer emission filter. Cells were analyzed in triplicate, average fluorescent units were calculated. Average background fluorescent units were measured from the control PCIneo transfection and were subtracted out from the average test fluorescent units.

For flow cytometry analysis, cells were washed twice with PBS, trypsinized (0.25% trypsin in 1 mM EDTA) and a single cell suspension was prepared for analysis (FACsort; Becton Dickinson, Franklin Lakes, N.J.). The percentage of GFP-positive cells was calculated by measuring GFP with a 530 nanometer band pass filter after excitation with a 488 nanometer line of an argon laser.

In Vitro Immunohistochemical Staining

Cells were plated in two-well chamber slides (4 cells per square centimeter). Cells were fixed with 100 percent ice-cold methanol for 10 minutes and immunostained with a polyclonal antibodies against rat TH (Pel-freeze, Rogers, Ariz.) at a dilution of 1:200 in 0.1 percent bovine serum albumin (BSA)-PBS. A rhodamine-conjugated goat anti-rabbit secondary antibody (Jackson Immunoresearch, West grove, Pa.) was used at a dilution of 1:200. For nuclear counterstaining, slides were incubated with 4′,6-di-amidino-2-phenylindole (DAPI, Sigma, St. Louis, Mo.) at a concentration of 1 microgram per milliliter after incubation with the secondary antibody.

Western Analysis

To obtain whole cells lysates, cells were washed twice in PBS and then scraped in lysis buffer (1 percent (v/v) NP-40, 0.5 percent (w/v) sodium deoxycholate, 0.1 percent (w/v) sodium dodecylsulfate (SDS) in PBS) containing 100 micrograms per milliliter of leupeptin (freshly prepared, Sigma). The lysate was transferred to a 1.5 milliliter microcentrifuge tube, passed through a syringe with a 21-gauge needle 3 times, and kept on ice. Phenylmethylsulfonyl fluoride (PMSF) was added to a final concentration of 570 micromolar. The lysate was incubated on ice for 30 minutes, spun down in a centrifuge at 15000 g for 20 minutes, and the supernatant was collected.

For protein quantification, a microdetermination protein kit (Micro Protein, Sigma, St. Louis, Mo.) was used. Ten micrograms of whole cells lysate was loaded onto a 4-20 percent acrylamide gradient gel (Bio-rad, Hercules, Calif.). After electrophoresis, the gel was electroeluted onto nitrocellulose at 70 volts for 1 hour. After transfer, the membrane was blocked with 5 percent non-fat dry milk in PBS for 1 hour, and incubated with 1:1000 rabbit polyclonal anti-TH antibodies diluted in TBS-T buffer (Tris-buffered saline, pH 8.0 with 0.05 percent Tween-20) overnight at 4 degrees Celsius. The following day, the blot was washed 3 times with TBS-T, incubated for 1 hour with 1:7500 HRP-labeled anti-rabbit secondary antibody (Santa Cruz Biotechnology, Santa Cruz, Calif.) diluted in TBS-T, washed with TBS-T 3 times, and detected with ECL chemiluminescent detection reagent (Amersham Pharmacia Biotech, Piscataway, N.J.) on chemiluminescent film (Hyperfilm ECL, Amersham).

High Pressure Liquid Chromatography (HPLC)

Cells were analyzed for L-DOPA production either after viral supernatant harvest (Phoenix cells) or after stable selection and expansion (rMSCs) as previously described in Schwarz E J et al., Hum Gene Ther 10:2539-2549, 1999. Briefly, HPLC was performed on an octadecylsilane (C18) column (Microsorb ShortOne; Rainin Instruments, Woburn, Mass.) with a mobile phase of 8.5 percent methanol in buffer (75 millimolar sodium phosphate, 10 micromolar disodium EDTA, and 1.4 millimolar octane sulfonic acid, pH 2.90) Coulometric detection (CoulochemII 5100A; ESA, Bedford, Mass.) was performed after sequential oxidation and reduction with the guard cell at a potential of 0.4 volts, electrode 1 at −0.25 volts, and electrode 2 at +0.35 volts. The rMSCs were washed 3 times in PBS, resuspended in Hank's balanced salt solution (HBSS), and 35 micrograms per milliliter L-tyrosine was added to the cells at time 0. A sample of the medium was collected and added to 75 millimolar perchloric acid and assayed for L-DOPA. After the experiment, the cells were counted on a hemacytometer.

The results of the experiments are now described.

Relative Promoter Strength in MSCs

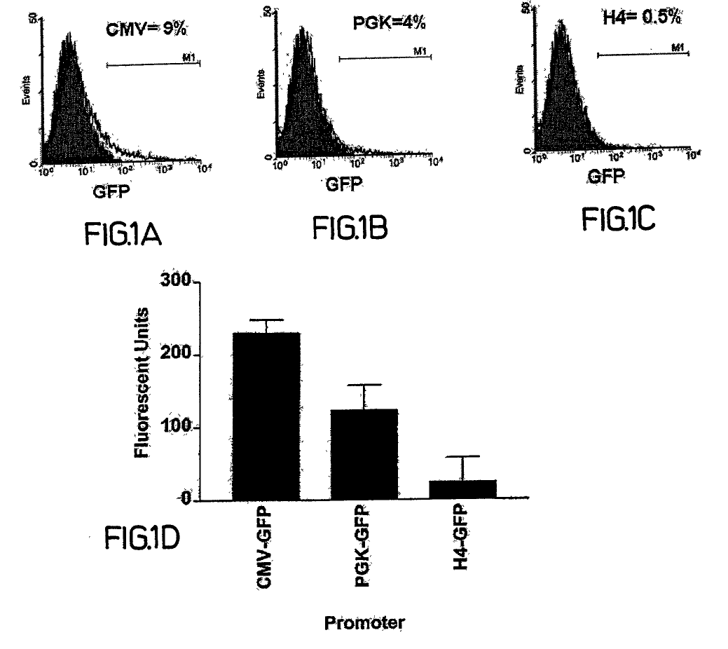

The relative promoter strengths of each of the plasmids created was tested in order to determine the effectiveness of each of the promoters selected for experimentation. Relative promoter strengths were analyzed approximately 72 hours after transient transfection of rMSCs with either CMV-GFP, PGK-GFP, or H4-GFP by assaying GFP fluorescence by either flow cytometry or with a fluorescent microtiter plate reader (FIG. 1). Nine percent of rMSCs transfected with CMV-GFP were GFP-positive. rMSCs transfected with PGK-GFP consistently expressed GFP in four percent of cells in culture. rMSCs transfected with H4-GFP expressed GFP at lower levels, typically 0.5 percent of cells in culture. Therefore, bicistronic constructs comprising either the CMV or PGK promoter and TIG were generated as described above.

TH Expression, GFP Expression, and L-DOPA Production in Phoenix Cells and Transduced MSCs

The Phoenix amphotropic packaging system was used to test expression of TIG or GFP in vectors and generate recombinant retrovirus in a short period of time. Recombinant retroviruses harvested from Phoenix cells transiently transfected with either LXSN-GFP, MSCV-GFP, pSIR-CMV-GFP, or pSIR-PGK-GFP, were used to transduce rMSCs using the method described above. Previously, helper virus production has not been detected in cells transduced with retroviruses produced in Phoenix cells (Limon A et al., Blood, 90:3316-3321, 1997). One special advantage of Phoenix cells is that viral supernatants can be collected from cells two days after transfection without the need for selection of the producer cells. Phoenix cells transfected with pSIR-CMV-GFP exhibited the highest amount of GFP expression. However, viral supernatants harvested from those cells were not able to transduce rMSCs, apparently because high levels of GFP can be toxic to some retroviral producer cells (Hanazano Y et al., Hum Gene Ther, 8:1313-1319, 1997). Phoenix cells transfected with the TH gene alone in the context of the MSCV promoter produced only one-fifth the amount of L-DOPA compared to cells transfected with both the TH and GC genes (TH-IRES-GC) in the same MSCV backbone, indicating the usefulness of the internal ribosome entry site in facilitating the production of both TH and GC from the same transcript and the necessity of GC in L-DOPA production.

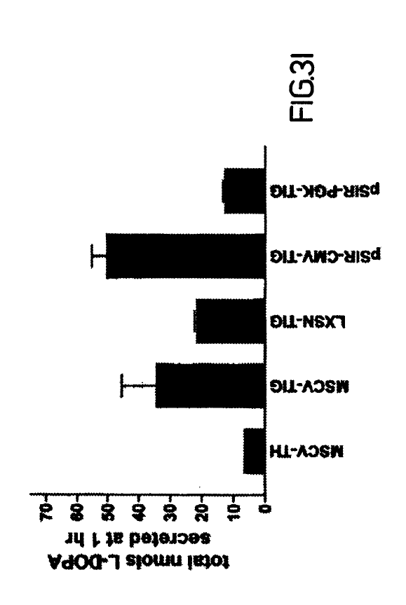

As shown in FIG. 4, approximately 8 to 10 percent of rMSCs transduced with retroviruses from LXSN-GFP (FIGS. 4A and 4B), MSCV-GFP (FIGS. 4C and 4D), or pSIR-PGK-GFP (FIGS. 4E and 4F) exhibited fluorescence due to GFP expression in rMSCs as analyzed by flow cytometry methods. However, none of the rMSCs transduced with retroviruses from Phoenix cells transfected with pSIR-CMV-GFP exhibited detectable GFP expression (FIGS. 4G and 4H).

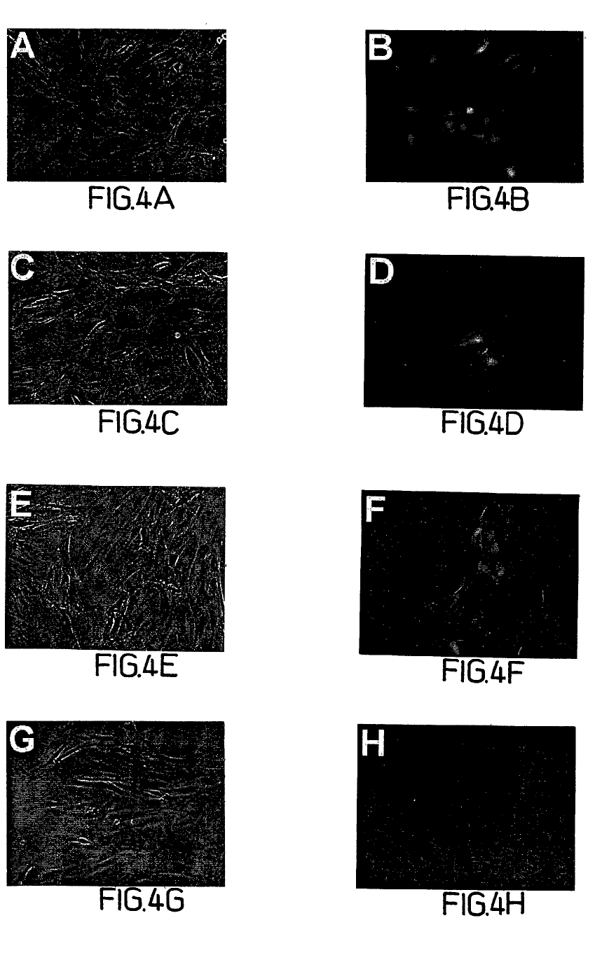

Fluorescence microscopy results indicated that approximately 63 percent of rMSCs stably transduced with LXSN-GFP (FIG. 5A) exhibited GFP fluorescence having a relative fluorescence intensity of 49.3. About 49 percent of rMSCs transduced with pSIR-PGK-GFP were GFP-positive and had a relative fluorescence intensity of 54.4. Roughly 59 percent of rMSCs transduced with MSCV-GFP (FIG. 5B) were GFP-positive, but this group displayed a low relative fluorescence intensity of 26.9. Expression of GFP in rMSCs transduced with pSIR-CMV-GFP was not detected.

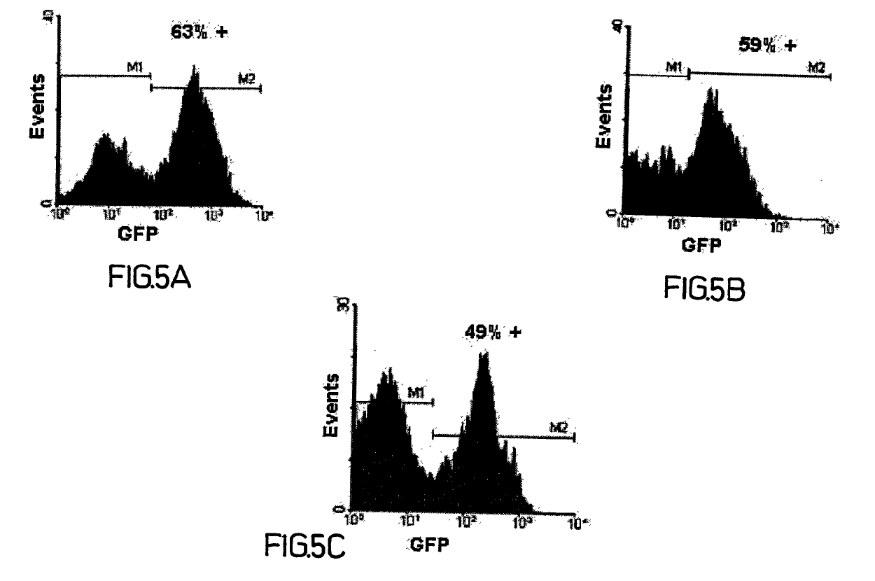

In determining how each of the constructs affected L-DOPA production, rMSCs were stably transduced as above, with either LXSN-TIG, MSCV-TIG, or pSIR-PGK-TIG. Due to the absence of a detectable level of GFP expression, rMSCs were not transduced with pSIR-CMV-TIG. The transduced rMSCs were analyzed for TH expression by immunostaining and Western analysis. FIGS. 6A and 6B illustrate rMSCs transduced with LXSN-TIG that were positive for TH immunostaining. rMSCs transduced with MSCV-TIG (FIGS. 6C and 6D) and pSIR-PGK-TIG (FIGS. 6E and 6D) also expressed TH by immunostaining.

Transduced cells were also analyzed for the presence of TH in whole cell lysates by Western analysis. Non-transduced, or wild-type, rMSCs did not have any endogenous levels of TH (FIG. 6G, lane 2; see also Schwarz E J et al., Hum Gene Ther, 10:2539-25491999). While all groups of transduced rMSCs expressed TH, rMSCs transduced with LXSN-TIG (FIG. 6G, lane 5) had higher levels of TH expression than those rMSCs transduced with MSCV-TIG (FIG. 6G, lane 4) or pSIR-PGK-TIG (FIG. 6G, lane 6). Cells transduced with MSCV-TH alone also expressed TH (FIG. 6G, lane 3), however, rMSCs transduced with both TH and GC had increased levels of TH (lane 4). This result is consistent with Leff et al., which previously reported that co-expression of GC with TH increased the expression of TH in the 9L gliosarcoma line (Leff S E et al., Exp Neurol, 151:249-264, 1998).

The effectiveness of the bicistronic sequence TH-IRES-GC (TIG) to produce the precursors to L-DOPA (TH and GC) was analyzed in transfected Phoenix cells. The results depicted in FIG. 31 illustrate packaging cells transfected with the control vector (MSCV vector comprising TH gene) produced 6.5 total nanomoles of L-DOPA in 1 hour, indicating that Phoenix cells have some endogenous levels of GC as reported by During et al., Gene Ther 5(6):820-7, 1998. However, when Phoenix cells were transfected with TIG in the same vector backbone (MSCV-TIG), approximately 34.4 nanomoles of L-DOPA were detected in the media after 1 hour, indicating that the bicistronic sequence is effective in providing the relevant precursors for L-DOPA production. Packaging cells transfected with LXSN-TIG, pSIR-CMV-TIG, and pSIR-PGK-TIG produced 21.7 nanomoles, 50.3 nanomoles, and 12.8 nanomoles L-DOPA, respectively. Relative amounts of L-DOPA production from each vector in the Phoenix cells closely paralleled GFP expression from the same vector backbones in Phoenix cells.

Levels of L-DOPA production was also measured by assaying the media in which transduced rMSCs were grown. When primary rMSCs were transduced with the retroviruses, high levels of GFP expression were exhibited in cells transduced with pSIR-PGK-GFP and LXSN-GFP, reflecting the higher expression in rMSCs of genes driven by the PGK promoter and the MMLV LTR. Surprisingly, the MSCV LTR, which is highly active in embryonic stem cells and hematopoietic stem cells (Conneally E, et al., Blood, 91(9):3487-3493, 1998), produced slightly lower levels of GFP expression in the adult rMSCs. L-DOPA production in the stably transduced rMSCs followed similar patterns as the rMSCs transduced with the GFP-containing constructs.

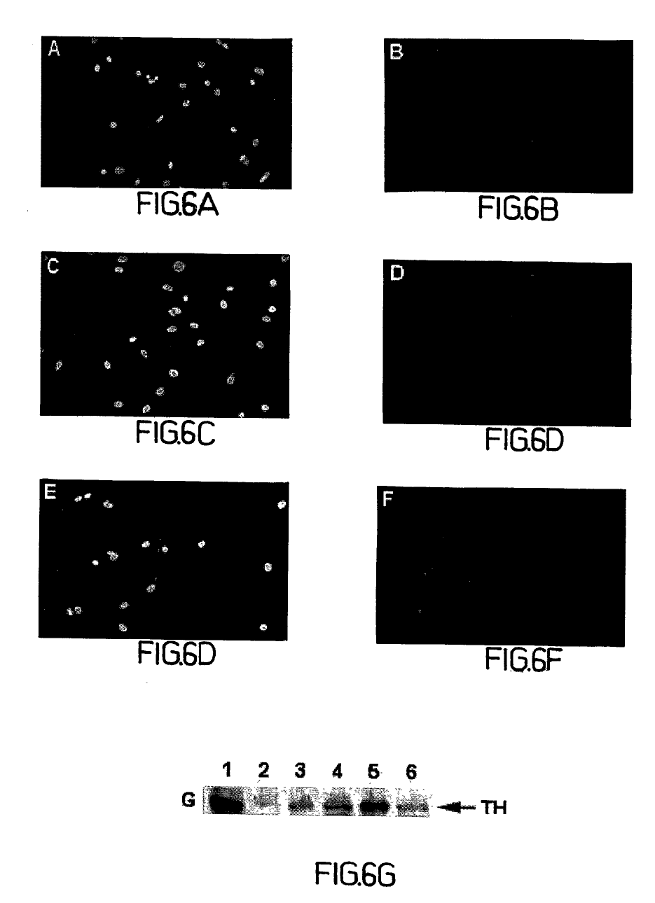

rMSCs transduced with TH alone did not spontaneously synthesize and secrete any L-DOPA due to the lack of the GC enzyme (Schwarz, et al., Hum Gene Ther, 10:2539-2549, 1999). As indicated in FIG. 7, rMSCs transduced with LXSN-TIG synthesized L-DOPA at a rate of 283±29.2 picomoles per 10⁶ cells per hour. rMSCs transduced with pSIR-PGK-TIG and MSCV-TIG produced similar amounts of L-DOPA, approximately 89.0±4.0 picomoles per 10⁶ cells per hour and 90.1 ±2.3 picomoles per 10⁶ cells per hour, respectively. Levels of L-DOPA production in the transduced cells with each construct closely paralleled levels of TH expression by Western analysis (FIG. 6G).

The production of L-DOPA in rMSCs transduced with pSIR-PGK-TIG was less than in previous experiments using the CMV promoter to drive TH and GC (Schwarz, et al., Hum Gene Ther, 10:2539-2549, 1999); however, the number of L-DOPA-producing MSCs transplanted into the striatum can be altered to target a known concentration of L-DOPA production. The low yields of adult MSCs transduced to synthesize L-DOPA can be overcome by simply plating MSCs at low-density (3 cells per square centimeter). When rMSCs stably transduced with LXSN-TIG, pSIR-PGK-TIG, and MSCV-TIG were plated at 3 cells per square centimeter, L-DOPA-producing cells increased in cell number over 1000-fold. MSCs plated at low-density may exhibit an enormous potential for self-renewal while maintaining their multi-potentiality (Colter D C et al., Proc Natl Acad Sci USA, 97(7):3213-3218, 2000).

Low-Density Expansion of L-DOPA-Producing MSCs

rMSCs stably transduced to produce L-DOPA as described previously above were tested for their ability to undergo a massive expansion when plated at low-density. rMSCs were plated at 3 cells per square centimeter in medium containing 200 micrograms per milliliter of G418 (Sigma, St. Louis, Mo.). Medium was replaced every 3-4 days, and cells were analyzed after 21 days in culture.

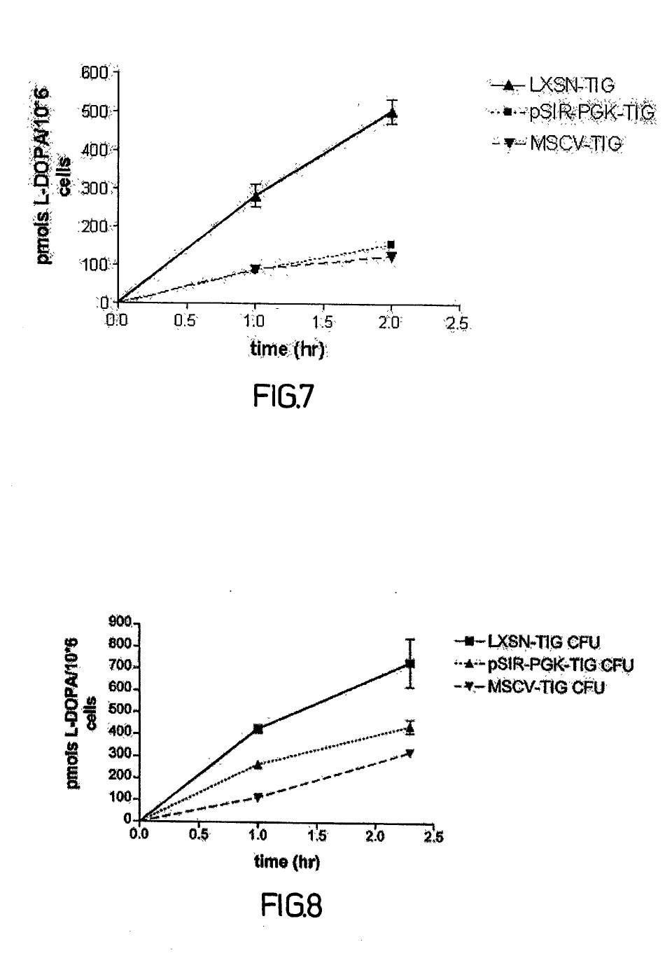

As shown in FIG. 8, transduced rMSCs plated at low-density continued to synthesize L-DOPA at levels similar to rMSCs grown under high-density conditions. Results indicated that rMSCs transduced with LXSN-TIG produced 424.8±18.4 picomoles per 10⁶ cells/hour of L-DOPA. Cells transduced with pSIR-PGK-TIG and MSCV-TIG produced 263.5±6.5 picomoles per 10⁶ cells per hour and 112.8±17.3 picomoles per 10⁶ cells per hour of L-DOPA, respectively. Furthermore, each group of transduced rMSCs increased in cell number over 1000-fold in a period of 21 days. rMSCs transduced with LXSN-TIG increased 1,434 fold±51, rMSCs transduced with pSIR-PGK-TIG increased 1,229 fold±72, and rMSCs transduced with MSCV-TIG increased 1,636 fold±293.

In summary, the results demonstrate that rMSCs can be transduced with either a self-inactivating retroviral vector or standard retroviral vectors containing a bicistronic sequence encoding therapeutic enzymes, TH and GC, necessary for L-DOPA synthesis in rMSCs. rMSCs stably transduced with the bicistronic constructs were able to synthesize L-DOPA and undergo at least a 1000-fold expansion in cell number by a single plating a low-density.

The possibility of using several different promoters and either a self-inactivating retrovirus or standard retroviruses to introduce into marrow stromal cells (MSCs) the two genes necessary for the cells to synthesize L-DOPA was examined. Rat MSCs (rMSCs) were transfected with plasmids containing a GFP reporter gene to assay the relative effectiveness of three different promoters: cytomegalovirus (CMV), mouse phosphoglycerate kinase-1 (PGK), and human histone 4 (H4). rMSCs transiently transfected with vectors containing the CMV or PGK promoters had the largest number of GFP positive cells (4-9% of cells). Self-inactivating retroviral vectors were then constructed using the PGK or CMV internal promoters to drive expression of either GFP or a bicistronic sequence containing the genes for human tyrosine hydroxylase type I (TH) and rat GTP cyclohydrolase I (GC) separated by an internal ribosome entry site (IRES). rMSCs were successfully transduced with both standard retroviral vectors and a self-inactivating vector containing the internal PGK promoter. Transduced rMSCs expressed GFP (49-63% of cells) or were able to synthesize and secrete L-DOPA (89-283 picomoles/10⁶ cells per hour). After transduced rMSCs were plated at low density (3 cells per square centimeter), the cells expanded over 1000-fold in 21 days, and the MSCs continued to produce L-DOPA (Schwarz et al. 1999. Hum. Gen. Ther. 10(15):2539-49).

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A method for transducing marrow stromal cells, said method comprising infecting marrow stromal cells with a vector which comprises a bicistronic coding region comprising a nucleic acid encoding tyrosine hydroxylase type I (TH) and a sequence encoding GTP cyclohydrolase I (GC), operably linked to a promoter/regulatory region, thereby transducing the marrow stromal cell.

2. The method of claim 1, wherein said vector is selected from the group comprising a virus and a plasmid.

3. The method of claim 2, wherein said virus is a retrovirus.

4. The method of claim 3, wherein said retrovirus is a self-inactivating retrovirus.

5. The method of claim 1, wherein said nucleic acid encoding TH and said nucleic acid encoding GC are separated by an internal ribosomal entry site (IRES).

6. The transduced marrow stromal cell of claim 1, wherein said marrow stromal cell is a human marrow stromal cell.

7. The transduced marrow stromal cell of claim 1, wherein said marrow stromal cell is a rat marrow stromal cell.

8. A method of treating Parkinson's disease, said method comprising administering to a patient marrow stromal cells transduced by the method of claim 1, wherein said administration of said marrow stromal cells alleviates symptoms of Parkinson's disease.

9. A method of treating a disease characterized by a deficiency in 3,4-dihydroxyphenylalanine (L-DOPA), said method comprising administering to a patient a marrow stromal cell transduced by the method of claim 1, wherein said administration of said marrow stromal cells regulates the production of L-DOPA causing alleviation of symptoms of said disease.

10. A method for producing exogenous L-DOPA, said method comprising transducing a marrow stromal cell by the method of claim 1 and expressing tyrosine hydroxylase type I (TH) and GTP cyclohydrolase I (GC) in said marrow stromal cell thereby producing exogenous L-DOPA.

11. A vector construct comprising a nucleic acid encoding TH and GC separated by an internal ribosomal entry site (IRES).

12. The vector construct of claim 11, wherein a promoter sequence is operably linked to the nucleic acids encoding TH and GC.

13. The vector construct of claim 12, wherein said promoter sequence is selected from the group consisting of cytomegalovirus promoter, phosphoglycerate kinase-1 promoter, or human histone H4.

14. The vector construct of claim 12, wherein said promoter sequence is cytomegalovirus promoter.

15. The vector construct of claim 12, wherein said promoter sequence is phosphoglycerate kinase promoter.

16. The vector construct of claim 13, wherein said vector is retroviral.

17. The vector construct of claim 16, wherein said vector is a self-inactivating retrovirus.

18. The vector construct of claim 11, wherein the vector is selected from the group consisting of murine leukemia viral vector (LXSN), murine stem cell viral vector (MSCV) and a self-inactivating retroviral vector, wherein said self-inactivating retroviral vector further comprises a promoter selected from the group consisting of cytomegalovirus and phosphoglycerate kinase promoter.
